(19) United States
(12) Patent Application Publication
(10) Pub. No.: US 2024/0009391 A1
(43) Pub. Date: Jan. 11, 2024

(54) System and Method for Pressure Sensor Based Empty Reservoir Detection for a Drug Delivery Device

(71) Applicant: Becton, Dickinson and Company, Franklin Lakes, NJ (US)

(72) Inventors: Steve Beguin, Rathdrum (IE); Sean McGrath, Artane (IE)

(21) Appl. No.: 17/859,643

(22) Filed: Jul. 7, 2022

(51) Int. Cl. A61M 5/172

(52) U.S. Cl. CPC A61M 5/172; A61M 2205/3337

(57) ABSTRACT

A drug delivery device includes a microcontroller, a pressure sensor, and/or a fluid pathway including a reservoir, a pump downstream of the reservoir, and/or a fluid line downstream of the pump. The reservoir may be configured to receive a fluid. The pump may be configured to deliver the fluid from the reservoir to the fluid line. The pressure sensor may configured to measure a pressure in the fluid pathway. The microcontroller may be programmed and/or configured to: receive, from the pressure sensor, the pressure measured in the fluid pathway; determine, based on the pressure measured in the fluid pathway, whether the reservoir is empty of the fluid; and/or control an output device to provide an indication associated with the determination of whether the reservoir is empty of the fluid.

### BACKGROUND

### Field

The present disclosure relates to a device and a method for pressure sensor based empty reservoir detection for a drug delivery device.

### Description of Related Art

Wearable medical devices, such as automatic injectors, have a benefit of providing therapy to a patient at a location remote from a clinical facility and/or while being worn discretely under the patient's clothing. A wearable medical device can be applied to the patient's skin and configured to automatically deliver a dose of a pharmaceutical composition within a predetermined time period after applying the wearable medical device to the patient's skin, such as after a 27 hour delay. After the device delivers the pharmaceutical composition to the patient, the patient may subsequently remove and dispose of the device.

In the context of fluid injection or infusion, some drugs may be prescribed to be administered with a tightly controlled dose regimen (e.g., a precise dose may be prescribed to be delivered with controlled timing, etc.). Injection devices typically allow for a controlled flowrate and dosing regimen based on volumetric dosing, where a system imposes a known volumetric displacement of the fluid being administered. Exemplary systems allowing for volumetric-based control of dosing are syringe pumps, oscillo-rotative pumps, systems with moving piston, peristaltic pumps, and membrane and diaphragm pumps.

A subset of these pumps work based on a “dosing chamber element” that is cyclically filled and emptied. A particularity of these systems is that the fluidic path upstream the pump may not be in direct communication with the path downstream the pump, allowing for different pressures to be established in these respective fluid paths.

An ability to detect when the device drug reservoir becomes empty may be desirable. It allows to notify the patient of successfully completed therapeutic treatment. This also allows to detect possible failure modes or when the reservoir is prematurely emptied, which can lead to under-dosing and may negatively impact the therapeutic treatment being administered. Precise and accurate detection of an empty reservoir may be particularly challenging in devices with flexible reservoirs, whereas syringe-type reservoirs may more easily exploit the linear movement of the syringe stopper.

### SUMMARY

Accordingly, provided are improved systems, devices, products, apparatus, and/or methods for empty reservoir detection for a drug delivery device.

According to some non-limiting embodiments or aspects, provided is a drug delivery device, including: a fluid pathway including a reservoir, a pump downstream of the reservoir, and a fluid line downstream of the pump, wherein the reservoir is configured to receive a fluid, and wherein the pump is configured to deliver the fluid from the reservoir to the fluid line; a pressure sensor configured to measure a pressure in the fluid pathway; and a microcontroller programmed and/or configured to: receive, from the pressure sensor, the pressure measured in the fluid pathway; determine, based on the pressure measured in the fluid pathway, whether the reservoir is empty of the fluid; and control an output device to provide an indication associated with the determination of whether the reservoir is empty of the fluid.

In some non-limiting embodiments or aspects, the pressure sensor is in the fluid pathway downstream of the pump.

In some non-limiting embodiments or aspects, the pressure sensor is in the fluid pathway upstream of the pump.

In some non-limiting embodiments or aspects, the microcontroller is programmed and/or configured to determine whether the reservoir is empty of the fluid by: comparing the pressure measured in the fluid pathway to a threshold pressure; and in response to the pressure measured in the fluid pathway satisfying the threshold pressure, determining that the reservoir is empty of the fluid.

In some non-limiting embodiments or aspects, the microcontroller is programmed and/or configured to determine whether the reservoir is empty of the fluid by: determining, based on the pressure measured in the fluid pathway, a rate of change associated with the pressure measured in the fluid pathway; comparing the rate of change associated with the pressure measured in the fluid pathway to a threshold rate of change; and in response to the rate of change associated with the pressure measured in the fluid pathway satisfying the threshold rate of change, determine that the reservoir is empty of fluid.

In some non-limiting embodiments or aspects, the pump includes a dosing chamber, and wherein the pump is configured to cyclically (i) pump the reservoir with the dosing chamber in fluid communication with the reservoir and not in fluid communication with the fluid line and (ii) fluidically connect the dosing chamber with the fluid line.

In some non-limiting embodiments or aspects, the microcontroller is further programmed and/or configured to: before fluidically connecting the dosing chamber with the fluid line, determine, based on the pressure measured in the fluid pathway, a baseline pressure, wherein the microcontroller is programmed and/or configured to determine whether the reservoir is empty based on the pressure measured in the fluid pathway and the baseline pressure.

In some non-limiting embodiments or aspects, the pressure sensor includes at least one of the following: an absolute pressure sensor, a differential pressure sensor, or any combination thereof.

According to some non-limiting embodiments or aspects, provided is a method for pressure sensor based empty reservoir detection for a drug delivery device including a fluid pathway including a reservoir configured to receive a fluid, a pump downstream of the reservoir, and a fluid line downstream of the pump, the method including: delivering, with the pump, the fluid from the reservoir to the fluid line; measuring, with a pressure sensor, a pressure in the fluid pathway; receiving, with a microcontroller, the pressure measured in the fluid pathway; determining, with the microcontroller, based on the pressure measured in the fluid pathway, whether the reservoir is empty of the fluid; and controlling, with the microcontroller, an output device to provide an indication associated with the determination of whether the reservoir is empty of the fluid.

In some non-limiting embodiments or aspects, the pressure sensor measures the pressure in the fluid pathway downstream of the pump.

In some non-limiting embodiments or aspects, the pressure sensor measures the fluid in the fluid pathway upstream of the pump.

In some non-limiting embodiments or aspects, determining whether the reservoir is empty of fluid includes: comparing the pressure measured in the fluid pathway to a threshold pressure; and in response to the pressure measured in the fluid pathway satisfying the threshold pressure, determining that the reservoir is empty of the fluid.

In some non-limiting embodiments or aspects, determining whether the reservoir is empty of fluid includes: determining, based on the pressure measured in the fluid pathway, a rate of change associated with the pressure measured in the fluid pathway; comparing the rate of change associated with the pressure measured in the fluid pathway to a threshold rate of change; and in response to the rate of change associated with the pressure measured in the fluid pathway satisfying the threshold rate of change, determining that the reservoir is empty of fluid.

In some non-limiting embodiments or aspects, the pump includes a dosing chamber, and delivering the fluid from the reservoir to the fluid line includes: cyclically (i) pumping the reservoir with the dosing chamber in fluid communication with the reservoir and not in fluid communication with the fluid line and (ii) fluidically connecting the dosing chamber with the fluid line.

In some non-limiting embodiments or aspects, the method further includes: before fluidically connecting the dosing chamber with the fluid line, determining, with the microcontroller, based on the pressure measured in the fluid pathway, a baseline pressure, wherein whether the reservoir is empty is determined based on the pressure measured in the fluid pathway and the baseline pressure.

In some non-limiting embodiments or aspects, the pressure sensor includes at least one of the following: an absolute pressure sensor, a differential pressure sensor, or any combination thereof.

According to some non-limiting embodiments or aspects, provided is a computer program product for pressure sensor based empty reservoir detection for a drug delivery device including a microcontroller, a pressure sensor, and a fluid pathway including a reservoir configured to receive a fluid, a pump downstream of the reservoir, and a fluid line downstream of the pump, the computer program product comprising at least one non-transitory computer-readable medium including program instructions that, when executed by the microcontroller, cause the microcontroller to: control the pump to deliver the fluid from the reservoir to the fluid line; control the pressure sensor to measure a pressure in the fluid pathway; receive the pressure measured in the fluid pathway; determine, based on the pressure measured in the fluid pathway, whether the reservoir is empty of the fluid; and control an output device to provide an indication associated with the determination of whether the reservoir is empty of the fluid.

In some non-limiting embodiments or aspects, the microcontroller controls the pressure sensor to measure the pressure in the fluid pathway downstream of the pump.

In some non-limiting embodiments or aspects, the pump includes a dosing chamber, wherein the pump delivers the fluid from the reservoir to the fluid line by cyclically (i) pumping the reservoir with the dosing chamber in fluid communication with the reservoir and not in fluid communication with the fluid line and (ii) fluidically connecting the dosing chamber with the fluid line.

In some non-limiting embodiments or aspects, the microcontroller controls the pressure sensor to measure the pressure in the fluid pathway upstream of the pump.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a drug delivery device according to one aspect or embodiment of the present application;

FIG. 2 is a perspective view of the drug delivery device of FIG. 1, with a top cover removed;

FIG. 3 is a schematic of the drug delivery device of FIG. 1;

FIG. 4 is a schematic of a system for pressure sensor based empty reservoir detection according to one aspect or embodiment of the present application;

FIG. 5 is a graph of an example pressure signature for the drug delivery device of FIG. 1;

FIG. 6 is a graph of another example pressure signature for the drug delivery device of FIG. 1;

FIG. 7 is a graph of example pressure signatures and motor current signatures for the drug delivery device of FIG. 1; and

FIG. 8 is a flow chart of a process for pressure sensor based empty reservoir detection for a drug delivery device according to one aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DESCRIPTION

Spatial or directional terms, such as “left”, “right”, “inner”, “outer”, “above”, “below”, and the like, are not to be considered as limiting as aspects or embodiments of the present disclosure can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

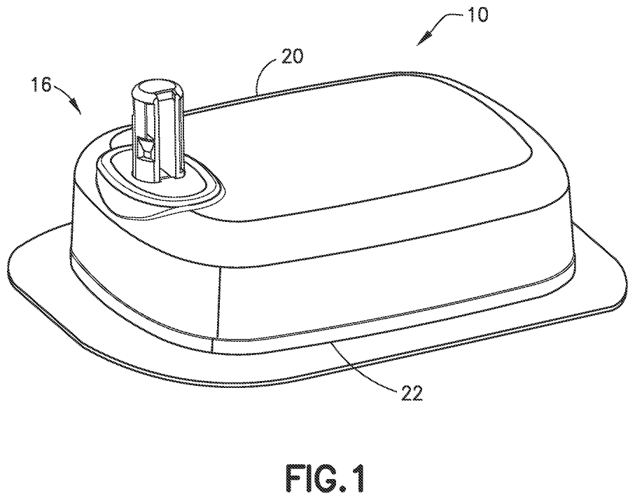

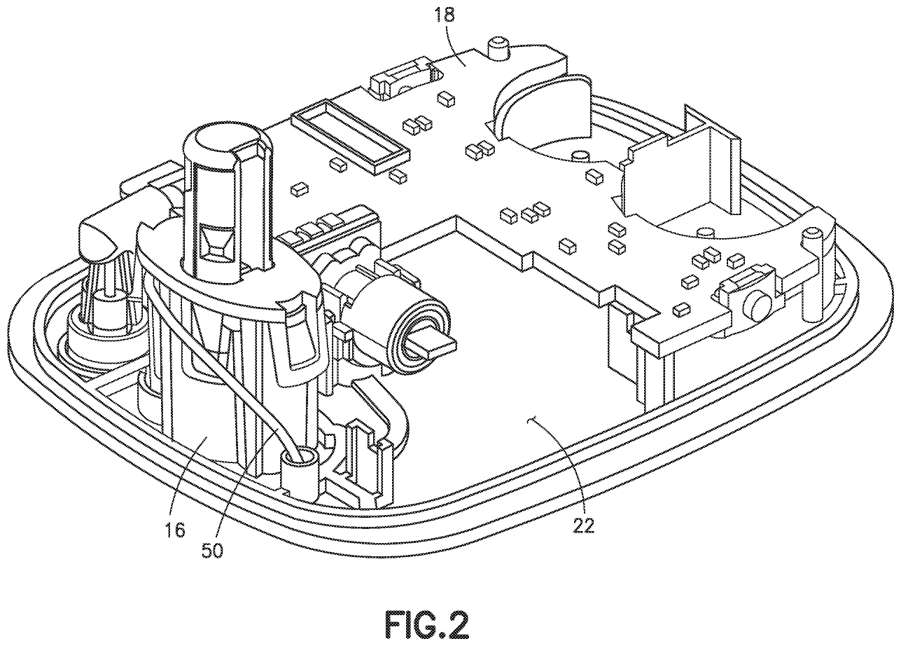

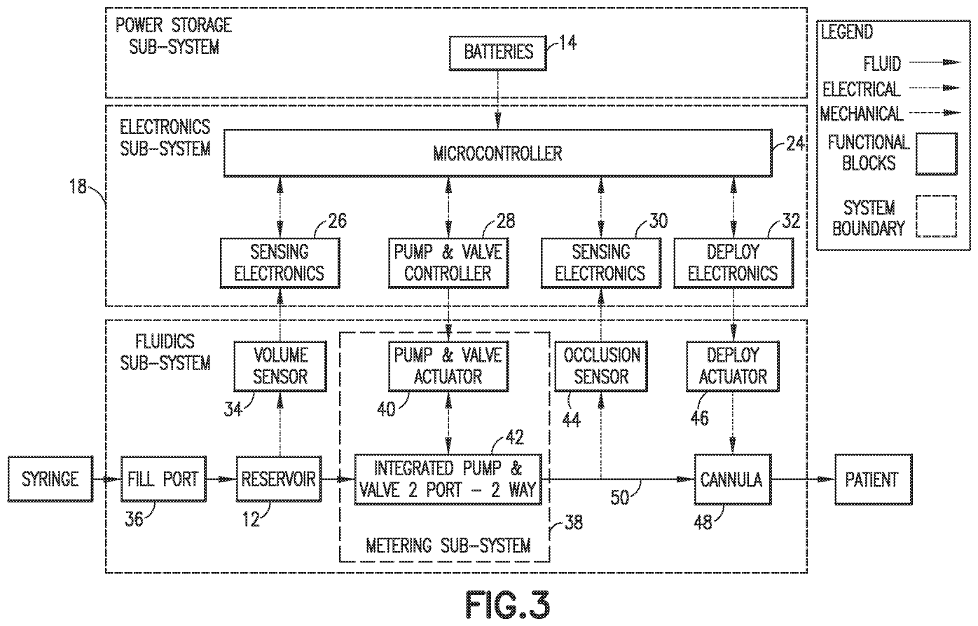

Referring to FIGS. 1-3, a drug delivery device 10 includes a reservoir 12, a power source 14, an insertion mechanism 16, control electronics 18, a cover 20, and a base 22. In one aspect or embodiment, the drug delivery device 10 is a wearable automatic injector, such as an insulin or bone marrow stimulant delivery device. The drug delivery device 10 may be mounted onto the skin of a patient and triggered to inject a pharmaceutical composition from the reservoir 12 into the patient. The drug delivery device 10 may be pre-filled with the pharmaceutical composition, or it may be filled with the pharmaceutical composition by the patient or medical professional prior to use.

The drug delivery device 10 is configured to deliver a dose of a pharmaceutical composition, e.g., any desired medicament, into the patient's body by a subcutaneous injection at a slow, controlled injection rate. Exemplary time durations for the delivery achieved by the drug delivery device 10 may range from about 5 minutes to about 60 minutes, but are not limited to this exemplary range. Exemplary volumes of the pharmaceutical composition delivered by the drug delivery device 10 may range from about 0 1 milliliters to about 10 milliliters, but are not limited to this exemplary range. The volume of the pharmaceutical composition delivered to the patient may be adjusted.

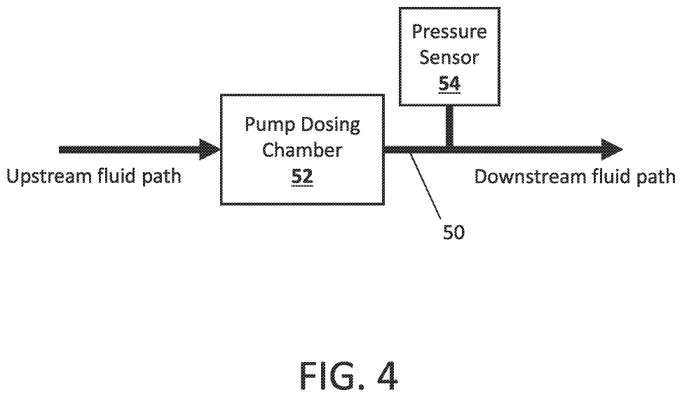

Referring again to FIGS. 1-3, in one aspect or embodiment, the power source 14 is a DC power source including one or more batteries. The control electronics 18 include a microcontroller 24, sensing electronics 26, a pump and valve controller 28, sensing electronics 30, and deployments electronics 32, which control the actuation of the drug delivery device 10. The drug delivery device 10 includes a fluidics sub-system that includes the reservoir 12, a volume sensor 34 for the reservoir 12, a reservoir fill port 36, and a metering system 38 including a pump and valve actuator 40 and a pump and valve mechanism 42. The fluidic sub-system may further include an occlusion sensor 44, a deploy actuator 46, a cannula 48 for insertion into a patient's skin, and a fluid line 50 in fluid communication with the reservoir 12 and the cannula 48. In one aspect or embodiment, occlusion sensor 44 includes a pressure sensor, such as pressure sensor 54 described in more detail herein with respect to FIG. 4. In one aspect or embodiment, the insertion mechanism 16 is configured to move the cannula 48 from a retracted position positioned entirely within the drug delivery device 10 to an extended position where the cannula 48 extends outside of the drug delivery device 10. The drug delivery device 10 may operate in the same manner as discussed in U.S. Pat. No. 10,449,292 to Pizzochero et al, incorporated herein by reference.

In one aspect or embodiment, a fluid pathway is formed by the reservoir 12, the pump and valve mechanism 42 downstream of the reservoir 12, and the fluid line 50 downstream of the pump and valve mechanism 42. For example, the reservoir 12 may be configured to receive a fluid, and the pump and valve mechanism 42 may be configured to deliver the fluid from the reservoir 12 to the fluid line 50.

Referring to FIG. 4, in one aspect or embodiment, pump and valve mechanism 42 includes a dosing chamber 52. The pump and valve mechanism 42 may be configured to cyclically (i) pump the reservoir 12 with the dosing chamber 52 in fluid communication with the reservoir 12 and not in fluid communication with the fluid line 50 and (ii) fluidically connect the dosing chamber 52 with the fluid line 50 (e.g., with the dosing chamber 52 not in fluid communication with the reservoir 12, etc.). For example, the pump and valve mechanism 42 may pump the reservoir 12 with the dosing chamber 52 in fluid communication with the reservoir 12 and not in fluid communication with the fluid line 50 to fill the dosing chamber 52 with the fluid from the reservoir 12, and the pump and valve mechanism 42 may connect the dosing chamber 52 with the fluid line 50 to empty the dosing chamber 52 of the fluid and deliver the fluid to the fluid line 50 (e.g., with the dosing chamber 52 not in fluid communication with the reservoir 12, etc.). In this way, the fluidic path upstream of the pump and valve mechanism 42 (e.g., upstream of the dosing chamber 52, etc.) may not be in direct fluid communication with the fluidic path downstream of the pump and valve mechanism 42, allowing for different pressures to be established in these respective upstream and downstream fluid paths.

Still referring to FIG. 4, the drug delivery device 10 may include a pressure sensor 54. The pressure sensor 54 may be configured to measure a pressure in the fluid pathway formed by the reservoir 12, the pump and valve mechanism 42 downstream of the reservoir 12, and the fluid line 50 downstream of the pump and valve mechanism 42. The pressure sensor 54 may be in the fluid pathway downstream of the pump and valve mechanism 42 and/or in the fluid pathway upstream of the pump and valve mechanism 42. For example, the pressure sensor 54 may be configured to measure the pressure in the fluid pathway downstream of the pump and valve mechanism 42 (e.g., downstream of the dosing chamber 52, etc.) and/or in the fluid pathway upstream of pump and valve mechanism 42 (e.g., upstream of the dosing chamber 52, etc.). The pressure sensor 54 may include at least one of the following: an absolute pressure sensor, a differential (e.g., gauge, etc.) pressure sensor, or any combination thereof. The pressure sensor 54 may be miniaturized, may have high resolution, may be cost effective, and/or may be optimized for low power consumption.

The microcontroller 24 may be programmed and/or configured to: receive, from the pressure sensor 54, the pressure measured in the fluid pathway; determine, based on the pressure measured in the fluid pathway, whether the reservoir 12 is empty of the fluid; and/or control an output device (e.g., a display, a light emitting diode (LED), a speaker, etc.) to provide an indication associated with the determination of whether the reservoir 12 is empty of the fluid. The output device may be included in and/or integrated with the drug delivery device 10 and/or the output device may be included in and/or integrated with an external device external to and in communication with (e.g., wireless and/or wireless communication, etc.) the drug delivery device 10, such as a remote computing device and/or a wireless controller (WC) 500 as discussed in U.S. Pat. No. 10,449,292 to Pizzochero et al, incorporated herein by reference.

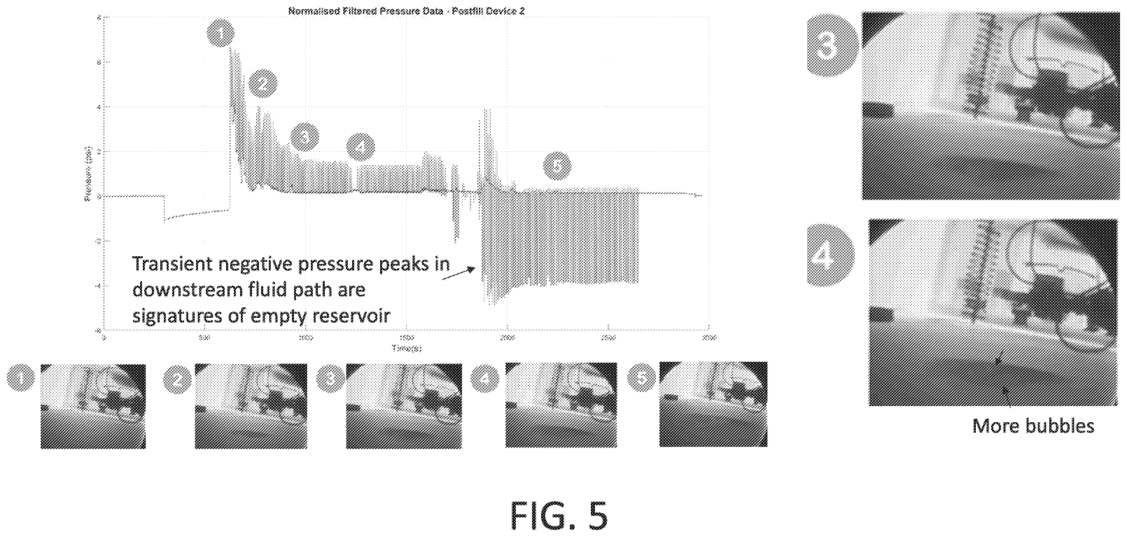

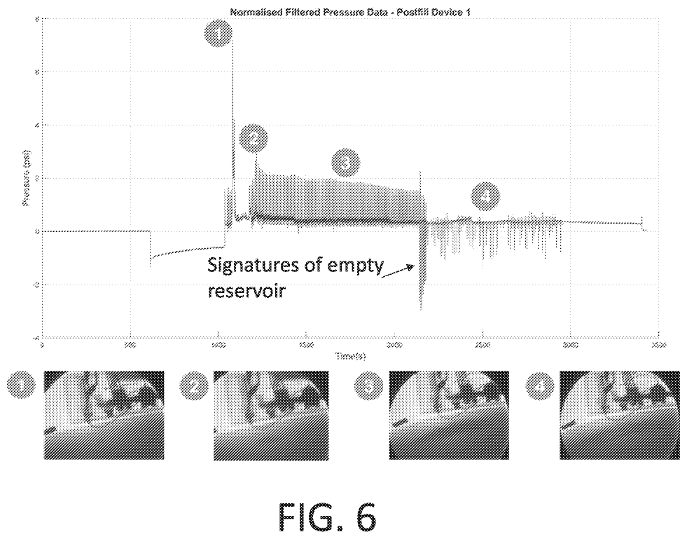

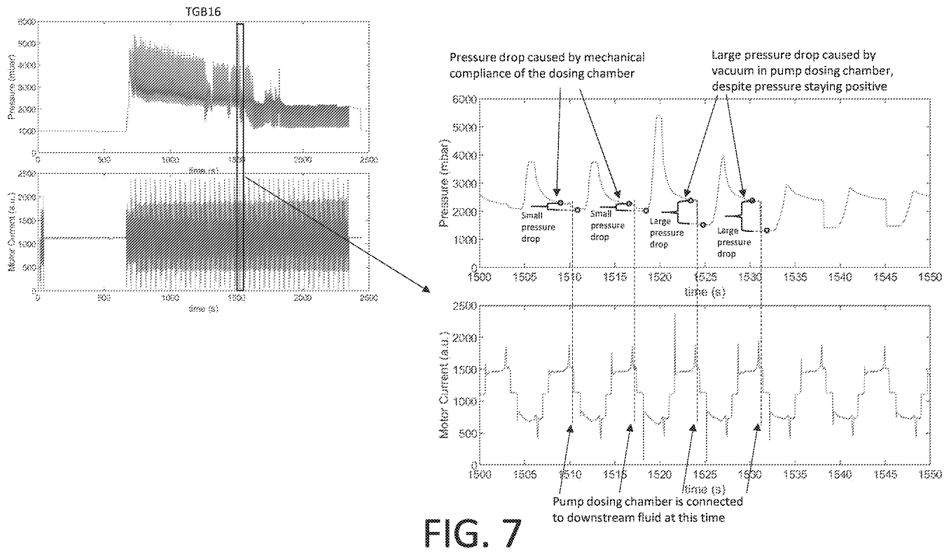

In this way, and referring also to FIGS. 5-7, when the dosing chamber 52 of the pump and valve mechanism 42 generates an empty volume after pumping the empty reservoir 12 and becomes in fluid communication with the downstream fluid path (e.g., the fluid line 50, etc.), the drug delivery device 10 may identify a pressure drop as a signature of an empty reservoir 12. For example, the vacuum pressure generated in the upstream fluid path upstream of the dosing chamber 52 may generate an identifiable pressure signature in the downstream fluid path downstream of the dosing chamber 52.

In one aspect or embodiment, the microcontroller 24 is programmed and/or configured to determine whether the reservoir 12 is empty of the fluid by: comparing the pressure measured in the fluid pathway to a threshold pressure; and in response to the pressure measured in the fluid pathway satisfying the threshold pressure, determining that the reservoir 12 is empty of the fluid. For example, the microcontroller 24 may compare a pressure drop reached when the dosing chamber 52 of the pump and valve mechanism 42 is connected to the downstream fluid path (e.g., to the fluid line 50, etc.) to the threshold pressure and, in response to the pressure drop satisfying the threshold pressure, determine that the reservoir 12 is empty of the fluid.

In one aspect or embodiment, the microcontroller 24 is programmed and/or configured to determine whether the reservoir 12 is empty of the fluid by: determining, based on the pressure measured in the fluid pathway, a rate of change associated with the pressure measured in the fluid pathway; comparing the rate of change associated with the pressure measured in the fluid pathway to a threshold rate of change; and in response to the rate of change associated with the pressure measured in the fluid pathway satisfying the threshold rate of change, determine that the reservoir 12 is empty of fluid. For example, the microcontroller 24 may compare the rate of change associated with the pressure measured in the fluid pathway (e.g., a rate of pressure increase, a rate of pressure decrease, a rate of pressure increase rate, a rate of pressure decrease rate, etc.) during pumping to the threshold rate of change and, in response to the measured rate of change satisfying the threshold rate, determine that the reservoir 12 is empty of the fluid.

In one aspect or embodiment, the microcontroller 24 is further programmed and/or configured to: before fluidically connecting the dosing chamber 52 with the fluid line 50, determine, based on the pressure measured in the fluid pathway, a baseline pressure; and determine whether the reservoir 12 is empty based on the pressure measured in the fluid pathway and the baseline pressure. For example, the measured pressure signature in the fluid pathway may be offset by a baseline pressure signature defined by a value of the pressure measured before connecting the dosing chamber 52 to the downstream fluid path.

In one aspect or embodiment, the indication associated with the determination of whether the reservoir 12 is empty of the fluid includes a notification of a successfully completed therapy or a notification of a failure mode and/or a prematurely empty reservoir. For example, the microcontroller 24 may be further programmed and/or configured to compare a time and/or a pump cycle at which the reservoir 12 is determined to be empty (e.g., using a clock and/or a counter of the microcontroller 24, etc.) to a threshold time and/or pump cycle, and determine based thereon that the therapy has been successfully completed or that a failure mode or prematurely empty reservoir is detected. As an example, in response to the time and/or pump cycle satisfying the threshold time and/or pump cycle, the microcontroller 24 may determine that the therapy has been successfully completed. As an example, in response to the time and/or pump cycle failing to satisfy the threshold time and/or pump cycle, the microcontroller 24 may determine that the drug delivery device 10 is in a failure mode and/or that the reservoir 12 has been prematurely emptied, which may lead to under-dosing and/or may negatively impact the therapeutic treatment being administered.

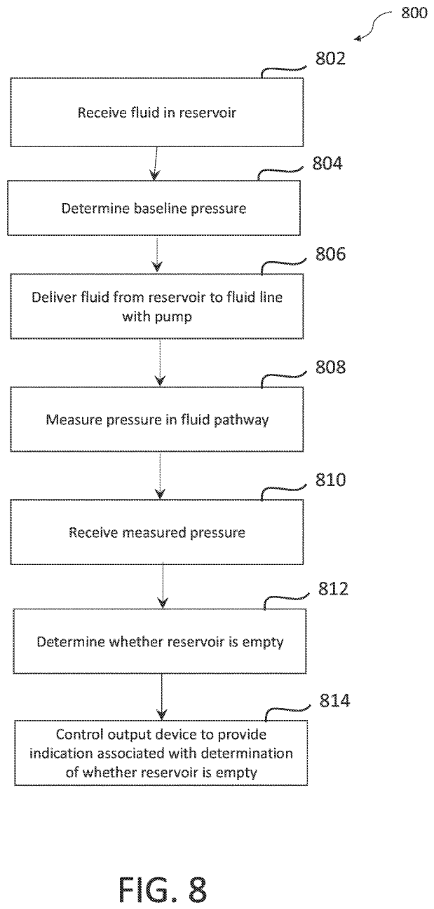

Referring to FIG. 8, in one aspect or embodiment, a process 800 for pressure sensor based empty reservoir detection for the drug delivery device 10 includes: receiving a fluid in the reservoir 12 (step 802); determining, with the microcontroller 24, based on a pressure measured in the fluid pathway (e.g., by pressure sensor 54, etc.), a baseline pressure (step 804); delivering, with the pump and valve mechanism 42, the fluid from the reservoir 12 to the fluid line 50 (step 806); measuring, with the pressure sensor 54, a pressure in the fluid pathway (step 808); receiving, with the microcontroller 24, the pressure measured in the fluid pathway (step 810); determining, with the microcontroller 24, based on the pressure measured in the fluid pathway, whether the reservoir 12 is empty of the fluid (step 812); and controlling, with the microcontroller 24, an output device to provide an indication associated with the determination of whether the reservoir 12 is empty of the fluid (step 814).

Accordingly, non-limiting embodiments or aspects of the present application may provide a drug delivery device having a low cost of manufacture, extremely low power consumption, a compact size, and/or low computing power requirements that may leverage potentially existing hardware in a pumping system and/or may be used with a flexible reservoir bag, rigid containers, and/or syringe-like containers.

Although aspects or embodiments have been described in detail for the purpose of illustration based on what is currently considered to be the most practical and preferred embodiments, it is to be understood that such detail is solely for that purpose and that aspects or embodiments of the present disclosure are not limited to the disclosed embodiments, but, on the contrary, are intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A drug delivery device, comprising:
a fluid pathway including a reservoir, a pump downstream of the reservoir, and a fluid line downstream of the pump, wherein the reservoir is configured to receive a fluid, and wherein the pump is configured to deliver the fluid from the reservoir to the fluid line;
a pressure sensor configured to measure a pressure in the fluid pathway; and
a microcontroller programmed and/or configured to:
receive, from the pressure sensor, the pressure measured in the fluid pathway;
determine, based on the pressure measured in the fluid pathway, whether the reservoir is empty of the fluid; and
control an output device to provide an indication associated with the determination of whether the reservoir is empty of the fluid.

2. The drug delivery device of claim 1, wherein the pressure sensor is in the fluid pathway downstream of the pump.

3. The drug delivery device of claim 1, wherein the pressure sensor is in the fluid pathway upstream of the pump.

4. The drug delivery device of claim 1, wherein the microcontroller is programmed and/or configured to determine whether the reservoir is empty of the fluid by:
comparing the pressure measured in the fluid pathway to a threshold pressure; and
in response to the pressure measured in the fluid pathway satisfying the threshold pressure, determining that the reservoir is empty of the fluid.

5. The drug delivery device of claim 1, wherein the microcontroller is programmed and/or configured to determine whether the reservoir is empty of the fluid by:
determining, based on the pressure measured in the fluid pathway, a rate of change associated with the pressure measured in the fluid pathway;
comparing the rate of change associated with the pressure measured in the fluid pathway to a threshold rate of change; and
in response to the rate of change associated with the pressure measured in the fluid pathway satisfying the threshold rate of change, determine that the reservoir is empty of fluid.

6. The drug delivery device of claim 1, wherein the pump includes a dosing chamber, and wherein the pump is configured to cyclically (i) pump the reservoir with the dosing chamber in fluid communication with the reservoir and not in fluid communication with the fluid line and (ii) fluidically connect the dosing chamber with the fluid line.

7. The drug delivery device of claim 6, wherein the microcontroller is further programmed and/or configured to:
before fluidically connecting the dosing chamber with the fluid line, determine, based on the pressure measured in the fluid pathway, a baseline pressure, wherein the microcontroller is programmed and/or configured to determine whether the reservoir is empty based on the pressure measured in the fluid pathway and the baseline pressure.

8. The drug delivery device of claim 1, wherein the pressure sensor includes at least one of the following: an absolute pressure sensor, a differential pressure sensor, or any combination thereof.

9. A method for pressure sensor based empty reservoir detection for a drug delivery device comprising a fluid pathway including a reservoir configured to receive a fluid, a pump downstream of the reservoir, and a fluid line downstream of the pump, the method comprising:
delivering, with the pump, the fluid from the reservoir to the fluid line;
measuring, with a pressure sensor, a pressure in the fluid pathway;
receiving, with a microcontroller, the pressure measured in the fluid pathway;
determining, with the microcontroller, based on the pressure measured in the fluid pathway, whether the reservoir is empty of the fluid; and
controlling, with the microcontroller, an output device to provide an indication associated with the determination of whether the reservoir is empty of the fluid.

10. The method of claim 9, wherein the pressure sensor measures the pressure in the fluid pathway downstream of the pump.

11. The method of claim 9, wherein the pressure sensor measures the fluid in the fluid pathway upstream of the pump.

12. The method of claim 9, wherein determining whether the reservoir is empty of fluid includes:
comparing the pressure measured in the fluid pathway to a threshold pressure; and
in response to the pressure measured in the fluid pathway satisfying the threshold pressure, determining that the reservoir is empty of the fluid.

13. The method of claim 9, wherein determining whether the reservoir is empty of fluid includes:
determining, based on the pressure measured in the fluid pathway, a rate of change associated with the pressure measured in the fluid pathway;
comparing the rate of change associated with the pressure measured in the fluid pathway to a threshold rate of change; and
in response to the rate of change associated with the pressure measured in the fluid pathway satisfying the threshold rate of change, determining that the reservoir is empty of fluid.

14. The method of claim 9, wherein the pump includes a dosing chamber, and wherein delivering the fluid from the reservoir to the fluid line includes:
cyclically (i) pumping the reservoir with the dosing chamber in fluid communication with the reservoir and not in fluid communication with the fluid line and (ii) fluidically connecting the dosing chamber with the fluid line.

15. The method of claim 14, further comprising:
before fluidically connecting the dosing chamber with the fluid line, determining, with the microcontroller, based on the pressure measured in the fluid pathway, a baseline pressure, wherein whether the reservoir is empty is determined based on the pressure measured in the fluid pathway and the baseline pressure.

16. The method of claim 9, wherein the pressure sensor includes at least one of the following: an absolute pressure sensor, a differential pressure sensor, or any combination thereof.

17. A computer program product for pressure sensor based empty reservoir detection for a drug delivery device comprising a microcontroller, a pressure sensor, and a fluid pathway including a reservoir configured to receive a fluid, a pump downstream of the reservoir, and a fluid line downstream of the pump, the computer program product comprising at least one non-transitory computer-readable medium including program instructions that, when executed by the microcontroller, cause the microcontroller to:
control the pump to deliver the fluid from the reservoir to the fluid line;
control the pressure sensor to measure a pressure in the fluid pathway;
receive the pressure measured in the fluid pathway;
determine, based on the pressure measured in the fluid pathway, whether the reservoir is empty of the fluid; and
control an output device to provide an indication associated with the determination of whether the reservoir is empty of the fluid.

18. The computer program product of claim 17, wherein the microcontroller controls the pressure sensor to measure the pressure in the fluid pathway downstream of the pump.

19. The computer program product of claim 18, wherein the pump includes a dosing chamber, wherein the pump delivers the fluid from the reservoir to the fluid line by cyclically (i) pumping the reservoir with the dosing chamber in fluid communication with the reservoir and not in fluid communication with the fluid line and (ii) fluidically connecting the dosing chamber with the fluid line.

20. The computer program product of claim 17, wherein the microcontroller controls the pressure sensor to measure the pressure in the fluid pathway upstream of the pump.
